(19) United States
(12) Patent Application Publication
(10) Pub. No.: US 2007/0224136 A1
(43) Pub. Date: Sep. 27, 2007

(54) Topical composition and method for improving skin characteristics

(75) Inventors: Bivash Ranjan Dasgupta, Hamden, CT (US); Meng Meng Zhang, Shanghai (CN); Qian Zhang, Shanghai (CN); Alexander Lips, New Canaan, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/389,720

(22) Filed: Mar. 27, 2006

(51) Int. Cl. A61K 8/27; A61K 8/29

(52) U.S. Cl. 424/59; 424/401; 977/926

(57) ABSTRACT

A topical composition and a method for improving skin characteristics are described. The topical composition comprises a mixture physical scatterers having a neat particle size from about 5 to less than about 100 nm and greater than about 300 nm to about 2 microns. The topical composition of the present invention, when applied, results in desirable skin lightening free of reddish and blotchy characteristics such that a 75 micron thick film of the same yields a red intensity (a) of less than ⁻1.

### FIELD OF THE INVENTION

The present invention is directed to a topical composition and a method for improving skin characteristics. More particularly, the present invention is directed to a composition that is, preferably, substantially free of physical scatterers having a neat particle size from about 100 nm to about 300 nm. The topical composition of the present invention, when applied to skin, unexpectedly, results in an even distribution of desirable skin lightening, free of reddish and blotchy characteristics.

### BACKGROUND OF THE INVENTION

Many people are concerned with the degree of pigmentation of their skin. For example, people with age spots or freckles often try to make such spots less pronounced. Others try to reduce skin darkening caused by exposure to sunlight or to lighten their natural skin color. To meet these needs, many attempts have been made to develop products that reduce pigment production in melanocytes (i.e., have a biological effect). However, the substances identified thus far tend to either have low efficacy, undesirable side effects, or both.

Still other consumers wish to apply an even or homogeneous layer to their skin with a composition that typically has a physical effect on the skin. Commercially available products of this nature, to date, are deficient since they tend to create a look that is very white, not white enough and/or reddish and blotchy.

There is increasing interest to develop a topical composition that has a biological and/or physical effect on the skin whereby the same, when applied, ensures an even and delightful lightening or whitening benefit free of a reddish characteristics. This invention, therefore, is directed to a topical composition (and a method for improving skin characteristics) whereby the composition is, preferably, substantially free of physical scatterers having a neat particle size from about 100 nm to about 300 nm. The topical composition of the present invention, when applied to skin, unexpectedly, results in an even distribution of desirable skin lightening, free of reddish and blotchy characteristics.

### ADDITONAL INFORMATION

Efforts have been disclosed for making topical compositions. In U.S. Pat. No. 6,875,425, cosmetic compositions with 4-substituted resorcinol are described.

Other efforts have been disclosed for making topical compositions. In U.S. Pat. Nos. 5,972,359, 5,997,890 and 6,001,377, skin care compositions for covering skin imperfections are described. The skin care compositions require particulate material with a neat particle size from about 100 nm to about 300 nm.

Even other efforts have been disclosed for making topical compositions. In U.S. Patent Application No. 2005/0036964 A1, cosmetic compositions for dark skins are disclosed.

None of the additional information above describes a topical composition that is suitable to impart an even and delightful lightening or whitening benefit free of reddish and blotchy characteristics.

### SUMMARY OF THE INVENTION

In a first aspect, the present invention is directed to a mixture of physical scatterers suitable for use in a topical skin composition, the mixture of scatterers preferably being substantially free of particles comprising a neat particle size from about 100 nm to about 300 nm and comprising a mixture of particles having neat particle sizes from about 5 to less than about 100 nm and greater than about 300 nm to about 2 microns.

In a second aspect, the present invention is directed to a topical composition comprising the mixture of physical scatterers of the first aspect of this invention.

In a third aspect, the present invention is directed to a method for improving skin characteristics with the composition of the second aspect of this invention.

Topical composition, as used herein, is meant to include cosmetic compositions suitable for use with humans and suitable to positively enhance a skin characteristic. Such a composition may generally be classified as leave-on or rinse-off and is meant to include hair care compositions like shampoos, conditioners or tonics; lipsticks; color cosmetics as well as a composition that reduces the impact of sebum, moisturizes and/or lightens. In a preferred embodiment, the topical composition is a leave-on composition applied directly to the skin. Neat particle size means the approximate diameter of the particles in powder form and prior to being combined with a carrier. Substantially free, as used herein, means 0.0 to less than about 0.3% by weight based on total weight of the composition. Preferably, however, the topical composition of this invention has less than about 0.25%, and most preferably, from about 0.0001 to less than about 0.1% by weight particles comprising a neat particle size from about 100 nm to about 300 nm. Scatterers, as used herein, mean particles that can reflect light and are suitable for use in a topical composition, where the scatterers are particles that can be compositions comprising, consisting essentially of, or consisting of, for example, particles of salt-like metal oxides and/or sulfides.

The composition of the present invention can be in the form of a liquid, lotion, cream, aerosol (i.e., made with standard chloroflourinated lower molecular weight hydrocarbons), gel, soap bar, ointment, or toner, or carried on a patch or face mask. The preferred composition is one that, at the very least, results in an even distribution of desirable skin lightening, free of reddish and blotchy characteristics on the skin, where skin is meant to include the skin on the face, neck, chest, back, arms, hands, legs and scalp. Blotchy is defined to mean marked with irregular or uneven shaped spots or blots as visually perceived. Free of reddish characteristics means having a red intensity (a) of less than about −1 when measuring the color of a 75 micron thick film of the topical composition with a Hunter Lab colorimeter (Lab Scan XE). All ranges identified herein are meant to implicitly include all ranges subsumed therein if, for example, reference to the same is not explicitly made.

### DETAILED DESCRIPTION OF THE PREFERRED EMBODIMENTS

The only limitation with respect to the type of physical scatterers that can be used in this invention is that the scatterers can be used in a topical composition suitable for use with humans. Types of physical scatterers that may be used can include salts and have a water solubility of under about 0.01%.

Preferred physical scatterers comprise (consist essentially of or consist of) particles that are +1, +2, +3 and +4 metal oxides and sulfides. Illustrative non-limiting examples of the types of physical scatterers that may be used in this invention include those with titanium, zinc, zirconium, silicon aluminum, iron and copper oxides and sulfides, mixtures thereof or the like, where the metal oxides are preferred, and especially, those comprising zinc, titanium, or both. In another preferred embodiment, the amount of physical scatterer used in the topical composition of this invention is from about 0.5 to about 20%, and preferably, from about 1.5 to about 15%, and most preferably, from about 2 to about 10% by weight, based on total weight of topical composition, and including all ranges subsumed therein.

As to the mixture of scatterers being preferably substantially free of particles comprising a neat particle size from about 100 nm to about 300 nm, the mixture preferably comprises scatterers having a neat particle size in the range from about 10 to about 75 nm, and most preferably, from about 15 to about 60 nm, including all ranges subsumed therein. Moreover, the mixture additionally, and preferably, comprises scatterers having a neat particle size from about 350 nm to about 1.5 microns, and most preferably, from about 400 nm to about 1 micron. The only limitation with respect to the ratio of physical scatterers used that are under 100 nm and over 300 nm is that the resulting topical composition made with the same has a red intensity (a) of less than about ⁻1, and preferably, less than about ⁻2. Typically, however the weight ratio of physical scatterers under 100 nm to physical scatterers over 300 nm is often from about 70:30 to about 30:70, and preferably, from about 60:40 to about 40:60, and most preferably, from about 45:55 to about 55:45, including all ratios subsumed therein. Moreover, it is typically desired for the refractive index of the physical scatterers employed herein (which preferably are pigmentary grade) to range from about 1.3 to about 3.2, and preferably, from about 1.4 to about 3.0, and most preferably, from about 1.5 to about 2.8, including all ranges subsumed therein and where refractive index is determined by conventional methods like those disclosed in J. A. Dean, Ed., Lange's Handbook of Chemistry, 14^{th} Ed., McGraw Hill, New York 1992.

It is particularly noted herein that the physical scatterers of this invention can be combined with optional additives, like fillers and pigments as long as the benefits of the invention described herein are not significantly reduced or impacted. Preferably, however, the physical scatterers of this invention at least consist essentially of particles (e.g., salt) as defined herein but are most preferably greater than 99.5% by weight salt particle, based on total weight of the physical scatterer. Such physical scatterers can be made via direct reactions of metals and sulfides or oxides, for example, and the same may also be obtained by supplying specifications to suppliers like Warner Jenkinson, U.S. Commetics or the like.

Furthermore, the mixture of physical scatterers selected for use in this invention preferably yield an opacity which is less than the opacity of the sum of the different scatterers used in the mixture. In an especially preferred embodiment, the opacity of the mixture of physical scatterers is less than about 45%, and most preferably, less than about 38%. In another especially preferred embodiment, the opacity of the mixture of scatterers is typically from about 0.5 to about 25%, and often, from about 1 to about 20%, and most often, from about 3 to about 10% less than the sum of the opacities for each physical scatterer used in the mixture, including all ranges subsumed therein.

Yet another benefit resulting from the use of the topical compositions of this invention is the fact that the same can offer a sun protection factor (SPF) of at least about 3, even in the absence of bona fide sunscreening agents like butylmethoxydibenzoyl methane, 2-ethylhexyl-p-methoxycinnamate, phenyl benzimidazole sulfonic acid, octocrylene, oxybenzone, cinnamate, octymethoxycinnamate or the like.

Commercially acceptable and conventional vehicles may be used, acting as diluants, dispersants or carriers for the mixture of physical scatterers of this invention in order to yield the desired topical (end use) composition. Therefore, the vehicle of the topical composition described herein may be aqueous-based, anhydrous or an emulsion whereby a water-in-oil or oil-in-water emulsion is generally preferred. If the use of water is desired, water typically makes up the balance of the topical composition, and preferably, makes up from about 5 to about 98%, and most preferably, from about 65 to about 90% by weight of the topical composition, including all ranges subsumed therein.

In addition to water, organic solvents may be optionally included to act or assist as carriers within the compositions of the present invention. Illustrative and non-limiting examples of the types of organic solvents suitable for use in the present invention include alkanols like methyl, ethyl and isopropyl alcohol, mixtures thereof or the like.

Other optional additives suitable for use include oils like isopropyl myristate, cetyl myristate, 2-octyldodecyl myristate, avocado oil, almond oil, olive oil, neopentylglycol dicaprate, lanolin, cod liver oil, sunflower oil, soybean oil, palm oil, cottonseed oil, menhaden oil, coconut oil, castor oil, safflower oil, peanut oil, pine oil, rice bran oil, mixtures thereof or the like. Typically, such oils assist in emulsifying the topical composition of this invention, and an effective amount is often used to yield a stable, and most preferably, water-in-oil emulsion.

Emollients may also be used, if desired, in the topical composition of the present invention. Alcohols like 1-hexadecanol (i.e., cetyl alcohol), behenyl alcohol and phenoxyethanol are often desired as are the emollients generally classified as silicone oils and synthetic esters. Silicone oils suitable for use include cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material in the inventive topical composition described herein include polyalkyl siloxanes, polyalkylaryl siloxanes and polyether siloxane copolymers. The essentially non-volatile polyalkyl siloxanes useful herein include, for example, polydimethylsiloxanes with viscosities of from about 5 to about 25 million centistokes at 25° C. Among the preferred non-volatile emollients useful in the present compositions are the polydimethylsiloxanes having viscosities from about 10 to about 400 centistokes at 25° C.

The ester emollients that may optionally be used are:
(1) alkenyl or alkyl esters of fatty acids having 10 to 20 carbon atoms.

Examples thereof include isoarachidyl neopentanoate, isononyl isonanonoate, oleyl myristate, oleyl stearate, and oleyl oleate.
(2) ether-esters such as fatty acid esters of ethoxylated fatty alcohols.
(3) polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) wax esters such as beeswax, spermaceti, stearyl stearate and arachidyl behenate.
(5) sterols esters, of which cholesterol fatty acid esters are examples.

While use of emollients is optional, the same can make up from about 0.1 to about 50% by weight of the topical composition, including all ranges subsumed therein.

Fatty acids having from about 10 to about 30 carbon atoms may also be included in the compositions of this invention. Illustrative of this category are pelargonic, lauric, myristic, palmitic, stearic, isostearic, hydroxystearic, oleic, linoleic, ricinoleic, arachidic, behenic, erucic acids, and mixtures thereof.

Humectants of the polyhydric alcohol type may also be employed in the topical composition of this invention. The humectant often aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant used may range anywhere from 0.2 to 15%, and preferably, from about 0.5 to about 7.0% by weight of the topical composition, including all ranges subsumed therein.

Thickeners may also be utilized as part of the cosmetically acceptable carrier in the topical composition of the present invention and they are often generally classified as carboxylic acid polymers, cross-linked polyacrylate polymers, polyacrylamide polymers or the like. Typical thickeners include cross linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose, polyacrylamide comprising thickeners (like Sepigel™305) and hydroxymethyl cellulose. Gums suitable for the present invention include guar, xanthan, magnesium aluminum silicate (Veegum), sclerotium, carrageenan, pectin and combinations of these gums. Amounts of the thickener may range from 0.0001 to 5%, usually from 0.001 to 1%, optimally from 0.01 to 0.5% by weight, based on total weight of the topical composition and including all ranges subsumed therein.

Collectively, water, solvents, silicones, esters, fatty acids, humectants and/or thickeners often constitute the cosmetically acceptable carrier in amounts from about 1 to about 99.5%, preferably from 80 to 98.5% by weight, based on total weight of the topical composition and including all ranges subsumed therein.

Various types of optional active ingredients may be used in the topical composition of the present invention. Actives are defined as skin benefit agents other than emollients and other than ingredients that merely improve the physical characteristics of the skin of the users of the composition. Although not limited to this category, general examples include anti-sebum ingredients such as talcs and silicas, as well as alpha-hydroxy acids, beta-hydroxy acids, poly-hydroxy acids, benzyl peroxide, zinc salts, and sunscreens.

A preferred hydroxy acid includes salicylic acid. Zinc pyrithione is an example of a preferred zinc salt useful in the topical composition of the present invention.

Sunscreens, while not required since the physical scatterers of this invention will result in a significant SPF in addition to skin lightening, may be used (in any desirable combination) and they include those materials commonly employed to block ultraviolet light. Illustrative compounds are PABA, cinnamate and salicylate. For example, avobenzophenone (Parsol 1789®) octyl methoxycinnamate and 2-hydroxy-4-methoxyl benzophenone (also known as oxybenzone) can be used. Octyl methoxycinnamate, 2-ethylhexyl-p-methoxycinnamate, and 2-hydroxy-4-methoxy benzophenone are all commercially available. Others which may be used include octocrylene, butylmethoxydibenzoyl methane and phenylbezimidazole sulfonic acid. The exact amount of sunscreen employed in the compositions can vary depending upon the degree of protection desired from the sun's UV radiation.

If desired, surfactants may be used in this invention and they typically have an HLB of greater than about 8. Such surfactants are preferably hydrophilic and can include cationic, zwitheronic, anionic, non-ionic or amphoteric surfactants, including mixtures thereof. Illustrative surfactants are described in McCutcheon's Detergents and Emulsifiers, North American Edition (1986), published by Allured Publishing Corporation, the disclosure of which is incorporated herein by reference. Surfactants like glycerol monostearate are often preferred.

Many cosmetic compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives for use in this invention are methyl paraben, propyl paraben, phenoxyethanol and benzyl alcohol. Preservatives will usually be employed in amounts ranging from about 0.1% to about 2% by weight of the topical composition.

Still other optional ingredients that may be used with the topical composition of this invention include chelators (like EDTA), anti-inflammatory agents (including the standard steroidal and non-steroidal types), vitamins, like niacinamide, anti-oxidants, exfoliants, dispersants (e.g., PEG-100 stearate), recorcinols and retinoids, including retinoic acid, retinal and retinyl esters.

The topical composition of the present invention is typically intended for use primarily as a product for topical application to human skin, especially as an agent for controlling the color or finish on the skin. Often, the topical composition of the present invention has a melting point from about 30° C. to about 45° C., a pH from about 2 to about 10.5, and most preferably, from about 4 to about 8, including all ranges subsumed therein.

When making the topical composition of the present invention, the desired ingredients are mixed in no particular order and usually at temperatures from about 70 to about 85° C. and under atmospheric pressure.

The packaging for the topical composition of this invention can be a bottle, tube, roll-ball applicator, propellant driven aerosol device, squeeze container or lidded jar.

The examples below are provided to illustrate the invention and are not intended to limit the scope of the claims.

### EXAMPLE 1

Topical compositions, consistent with the description herein, were made with the following ingredients.

| Ingredient | Weight Percent |
|---|---|
| TiO₂ (60 nm) | 1 |
| TiO₂ (500 nm) | 1.2 |
| Humectant | 6 |
| Silicone Oil | 3.5 |
| Thickener | 1.25 |
| Stearic acid | 1 |
| Surfactant (non-ionic) | 1 |
| Gum | 0.2 |
| Water | Balance |

The compositions were made by adding the gum to water, followed by the additional ingredients. Mixing was achieved (within about 30 minutes) with a standard mixing device set from about 250 to about 300 rpm. The mixing temperature was about 80° C. The resulting mixed composition was transferred to a homogenizer set at about 3000 rpm. The mixed composition was homogenized for about 30 minutes at 80° C. to produce the superior topical composition of this invention.

### EXAMPLE 2

Films of compositions similar to those made in Example 1 (75 microns) were prepared on draw down cards, and the red intensity of the same was assessed with a Hunter Lab Colorimeter (Lab Scan XE). The compositions made according to this invention had a radiant and unexpected red intensity of about ⁻2. These compositions were compared to control compositions (commercially sold under the names Olay White Radiance Protection Cream and Olay Total Effects) having from about 0.3 to about 2% by weight TiO₂ particles in the 100 nm to 300 nm size range. The red intensity of the controls was significantly less desirable (i.e., about ⁻0.2) than the red intensity of the compositions made according to this invention.

### EXAMPLE 3

Approximately 50 panelists applied to their skin a film of the topical composition made according to this invention. The same panelists also applied the control compositions described in Example 2. Almost all panelists concluded that the controls created a film that was blotchy and not radiant when applied wherein such negative characteristics were not observed when applying the topical compositions made according to this invention.

What is claimed is:

1. A mixture of physical scatterers comprising:
(a) first particles comprising a neat particle size from about 5 to about 100 nm; and
(b) second particles comprising a neat particle size from greater than about 300 nm to about 2 microns,
the mixture of physical scatterers being suitable for use in a topical composition and the mixture of physical scatterers having an opacity which is less than a sum of opacity for each physical scatterer within the mixture of physical scatterers.

2. The mixture of physical scatterers according to claim 1 wherein physical scatterers comprising the first particles comprise a neat particle size from about 10 to 75 nm and physical scatterers comprising the second particles comprise a neat particle size from about 350 nm to about 1.5 microns.

3. The mixture of physical scatterers according to claim 1 wherein the physical scatterers comprising the first particles and the physical scatterers comprising second particles are at a weight ratio from about 70:30 to about 30:70.

4. The mixture of physical scatterers according to claim 1 wherein the physical scatterers comprise particles that are +1, +2, +3 and +4 metal oxides or metal sulfides or both.

5. The mixture of physical scatterers according to claim 4 wherein the metal oxides and sulfides comprise zinc, zirconium, silicon, aluminum, iron, copper or mixtures thereof.

6. The mixture of physical scatterers according to claim 4 wherein the particles are titanium dioxide, zinc oxide or mixtures thereof.

7. A topical composition comprising:
(a) a mixture of physical scatterers comprising physical scatterers comprising first particles having a neat particle size from about 5 to about 100 nm and physical scatterers comprising second particles comprising a neat particle size from greater than about 300 nm to about 2 microns; and
(b) a cosmetically acceptable vehicle,
wherein about a 75 micron film of the topical composition has a red intensity of less than about ⁻1.

8. The topical composition according to claim 7 wherein the physical scatterers comprising the first particles have a neat particle size from about 10 to 75 nm and the physical scatterers comprising the second particles have a neat particle size from about 350 nm to about 1.5 microns.

9. The topical composition according to claim 7 wherein a 75 micron film of the topical composition has a red intensity of less than about ⁻2.

10. The topical composition according to claim 7 wherein the mixture of physical scatterers has an opacity which is less than a sum of opacities for each physical scatterer within the mixture of physical scatterers.

11. The topical composition according to claim 7 wherein the physical scatterers comprising the first particles and the physical scatterers comprising second particles are at a weight ratio from about 70:30 to about 30:70.

12. The topical composition according to claim 10 wherein the mixture of physical scatterers has an opacity of less than about 40%.

13. The topical composition according to claim 10 wherein the opacity of the mixture of physical scatterers is from about 0.5 to about 25% less than the sum of the opacities for each physical scatterer within the mixture of physical scatterers.

14. The topical composition according to claim 7 wherein the physical scatterers comprise particles that are +1, +2, +3 and +4 metal oxides or metal sulfides or both.

15. The topical composition according to claim 14 wherein the metal oxides and sulfides comprise zinc, zirconium, silicon, aluminum, iron, copper or mixtures thereof.

16. The topical composition according to claim 14 wherein the particles are titanium dioxide, zinc oxide or mixtures thereof.

17. A method for treating skin comprising the steps of:
(a) obtaining a topical composition according to claim 7; and
(b) applying the topical composition to the skin.
